(19) United States
(12) Patent Application Publication
(10) Pub. No.: US 2024/0211541 A1
(43) Pub. Date: Jun. 27, 2024

(54) METHOD AND SYSTEM FOR PROVIDING LABELED IMAGES FOR SMALL CELL SITE SELECTION

(71) Applicants: Soufiane BELHARBI, Montareal (CA); Eric GRANGER, Montareal (CA); Aydin SARRAF, Pirrefonds (CA)

(72) Inventors: Soufiane Belharbi, Montreal (CA); Eric Granger, Montreal (CA); Aydin Sarraf, Pierrefonds (CA)

(73) Assignee: TELEFONAKTIEBOLAGET LM ERICSSON (PUBL), Stockholm (SE)

(21) Appl. No.: 18/555,136

(22) PCT Filed: Apr. 15, 2021
(86) PCT No.: PCT/IB2021/053139

§ 371 Date: Oct. 12, 2023

(51) Int. Cl. G06F 18/214; G06T 7/11; G16H 30/40

(52) U.S. Cl. CPC G06F 18/214; G06T 7/11; G16H 30/40; G06T 2207/20081

(57) ABSTRACT

The disclosure relates to a method for training a model for labeling images. The method comprises obtaining a first dataset of labeled images, the first dataset of labeled images being selected from a pool of images presenting different views of portions of geographical areas; selecting, from the pool of images, a second dataset of images for pseudo labeling, different from the first dataset; feeding the second dataset of images into the model and obtaining as output of the model the second dataset of images with pseudo-labels; combining the first dataset of labeled images with the second dataset of images with pseudo-labels into a third dataset; training the model with the third dataset; and testing the model with a fourth dataset of labeled images and, upon determining that a requested performance is met, storing the model, or, upon determining that the requested performance is not met, executing the method again.

### TECHNICAL FIELD

The present disclosure relates to the selection of small cell sites and providing labeled images therefor.

### BACKGROUND

A major challenge in small cell installations is to find suitable small cell hosting structures. An efficient small cell installation framework is particularly important for 5G deployment because 5G demands small cells to be deployed at a faster pace and at a greater density. Many factors determine the suitability of a hosting structure such as power source availability, backhaul connectivity, environmental conditions, and local zoning requirements. For example, a location such as a street corner may meet all the technical requirements, but the municipal aesthetic regulations may prohibit the installation of small cells at street corners.

Therefore, there is a need for a method for determining suitable locations for small cells installation.

### SUMMARY

There is provided a method for training a model for labeling images. The labeled images may be used for small cell site selection. The method comprises obtaining a first dataset of labeled images, the first dataset of labeled images being selected from a pool of images presenting different views of portions of geographical areas. The method comprises selecting, from the pool of images, a second dataset of images for pseudo labeling, different from the first dataset. The method comprises feeding the second dataset of images into the model and obtaining as output of the model the second dataset of images with pseudo-labels. The method comprises combining the first dataset of labeled images with the second dataset of images with pseudo-labels into a third dataset. The method comprises training the model with the third dataset. The method comprises testing the model with a fourth dataset of labeled images and, upon determining that a requested performance is met, storing the model, or, upon determining that the requested performance is not met, executing the method again.

There is provided a method for obtaining labeled images. The method comprises providing a dataset of images presenting different views of portions of geographical areas to a trained model, the model being trained using a weakly-supervised technique and receiving as input for the training a dataset of labeled images and a dataset of images previously labeled by the model in training. The method comprises receiving a dataset of images labeled by the model identifying, at pixel level, physical objects pertaining to predefined classes of objects suitable for small cell site selection.

There is provided a system operative to train a model for labeling images comprising processing circuits and a memory. The memory contains instructions executable by the processing circuits whereby the system is operative to obtain a first dataset of labeled images, the first dataset of labeled images being selected from a pool of images presenting different views of portions of geographical areas. The system is operative to select, from the pool of images, a second dataset of images for pseudo labeling, different from the first dataset. The system is operative to feed the second dataset of images into the model and obtain as output of the model the second dataset of images with pseudo-labels. The system is operative to combine the first dataset of labeled images with the second dataset of images with pseudo-labels into a third dataset. The system is operative to train the model with the third dataset. The system is operative to test the model with a fourth dataset of labeled images and, upon determining that a requested performance is met, storing the model, or, upon determining that the requested performance is not met, executing the method again.

There is provided a system operative to obtain labeled images comprising processing circuits and a memory. The memory contains instructions executable by the processing circuits whereby the system is operative to provide a dataset of images presenting different views of portions of geographical areas to a trained model, the model being trained using a weakly-supervised technique and receiving as input for the training a dataset of labeled images and a dataset of images previously labeled by the model in training. The system is operative to receive a dataset of images labeled by the model identifying, at pixel level, physical objects pertaining to predefined classes of objects suitable for small cell site selection.

There is provided a non-transitory computer readable media having stored thereon instructions for training a model for labeling images. The instructions may comprise any of the steps described herein.

There is provided a non-transitory computer readable media having stored thereon instructions for obtaining labeled images. The instructions may comprise any of the steps described herein.

The methods and systems provided herein present improvements to the way suitable locations for small cell installation can be determined.

### BRIEF DESCRIPTION OF THE DRAWINGS

FIGS. 1 and 2 are flowcharts illustrating the training process.

FIG. 3 is a schematic illustration of hybrid architecture for classification and segmentation of images.

FIG. 4 is a schematic illustration of a proposed active learning framework with a weak annotator.

FIG. 5 is a schematic illustration of a how samples are selected for pseudo-labeling.

FIG. 6 is a flowchart illustrating the inference process.

FIG. 7 is a schematic illustration of a sample image with annotated regions: black for vegetation, dotted for building, and hatched (diagonal stipes) for poles.

FIG. 8 is a graph illustrating average Dice index of different methods using the Cityscapes test set.

FIG. 9 is a graph illustrating average Dice index over the pseudo-labeled samples in each active learning (AL) round.

FIG. 10 is a graph illustrating the impact of the parameter lambda on the area under the curve (AUC) of the Dice index.

FIG. 11a is a flowchart of a method for training a model for labeling images.

FIG. 11b is a flowchart of a method for obtaining labeled images.

FIG. 12 is a schematic illustration of a virtualization environment in which different methods, systems and apparatuses described herein can be deployed.

### DETAILED DESCRIPTION

Various features will now be described with reference to the drawings to fully convey the scope of the disclosure to those skilled in the art.

Sequences of actions or functions may be used within this disclosure. It should be recognized that some functions or actions, in some contexts, could be performed by specialized circuits, by program instructions being executed by one or more processors, or by a combination of both.

Further, computer readable carrier or carrier wave may contain an appropriate set of computer instructions that would cause a processor to carry out the techniques described herein.

The functions/actions described herein may occur out of the order noted in the sequence of actions or simultaneously. Furthermore, in some illustrations, some blocks, functions or actions may be optional and may or may not be executed; these may be illustrated with dashed lines.

To determine the suitability of a location for a small cell installation, visual inspection is often required. Thanks to the availability of three dimensional (3D) images of street views, potential locations can be inspected by an analysis of the 3D images. The image analysis for small cell installation is not limited to outdoor scenes and it can be extended to indoor scenes if 3D images are available from the indoor areas. Some examples of outdoor small cell hosting structures are poles, traffic signals, billboards, bus shelters, water towers, and building mounts. Each of these regions of interests (ROIs) can be further divided into more categories. For example, poles can be further categorized into streetlights vs utility poles, wooden vs metal poles, crowded vs uncrowded poles, tall vs short poles, etc.

Herein, only visually identifiable characteristics of ROIs are of interest. Currently, classification and segmentation of ROIs in outdoor/indoor scenes are typically done through supervised learning methods.

In supervised learning methods, the global and pixel-level annotation of images must be provided manually, which is quite cumbersome and time consuming. Convolutional neural networks (CNNs) can be trained using image data with global annotations to classify images. In addition, interpretation methods like class activation maps (CAMs) can also provide saliency maps that highlight image regions. However, these CAMs are lower resolution, and often provide poor object localization.

There is a need for a new learning method that can provide a higher level of localization and segmentation accuracy with far less pixel-wise annotations than a fully supervised technique. The pixel-wise annotation cost can be reduced by introducing a new weakly supervised learning method. The new learning method can train on millions of pixel-wise unlabeled N-dimensional (ND) images—here, dimension refers to the physical dimension, i.e. N is typically between 2 (x, y) and 4 (x, y, z, t)—to design a semantic segmenter for radio frequency (RF) planners. A semantic segmenter, in the present context, is defined as a pre-trained classifier that can classify images and can also localize, and thereby segment, the ROIs in images. The semantic segmenter which is pretrained by the novel weakly supervised learning method described herein can be marketed as a standalone software product.

The method described herein proposes to combine an active learning (AL) method with a pseudo-labeling method (which is a semi-supervised learning technique) to improve the performance over current active learning techniques. Herein, pseudo-labelling, pseudo-policy and pseudo-annotation may be used interchangeably. Moreover, the proposed method is capable of joint classification and semantic segmentation of input images. In this work, pixel-wise annotation is provided progressively, using an active learning framework and a pseudo-labeling framework. The pseudo-labeling framework is based on a deep convolutional architecture (segmentation head). This architecture allows supervised learning of a model that can accurately classify and segment input images, thereby producing high resolution salient regions of interest (ROI) that can be viewed as accurate segmentations.

The method allows classifying an input image and locating the ROIs within this image that are associated with a plurality of class predictions. These ROIs are indicated using class activation maps (CAMs). Using such spatial information allows to assign a label to each pixel, which provides a segmentation for the image. With this method, active learning of pixel-wise labels allows improving the maps and thus segmentations that can be obtained.

Semantic segmentation is important in identifying and delineating the contours of ROIs. The segmented ROIs can then be shown to an expert or another computer program in order to find candidate locations for small cell installations or to get a better understanding of the region to design a better radio frequency propagation model.

The input images may be taken from Google™ street map where a set of streetlight poles have been labeled by a human or by other supervision techniques, such that the geodetic locations and/or orientations of the streetlights are known. Image augmentation proceeds to create a plurality of annotated images by generating randomized translational and rotational viewings of the labeled images.

As an example, a polar coordinate angular rotation vector of (r, Θ) with 30 values of r and 36 values for Θ results can generate over 1000 images. However, only a smaller subset of these images is sufficient to significantly improve algorithmic accuracy. That subset may be 1% or 10% of the potential set of generated images, selected to achieve a specified customer defined service level agreement.

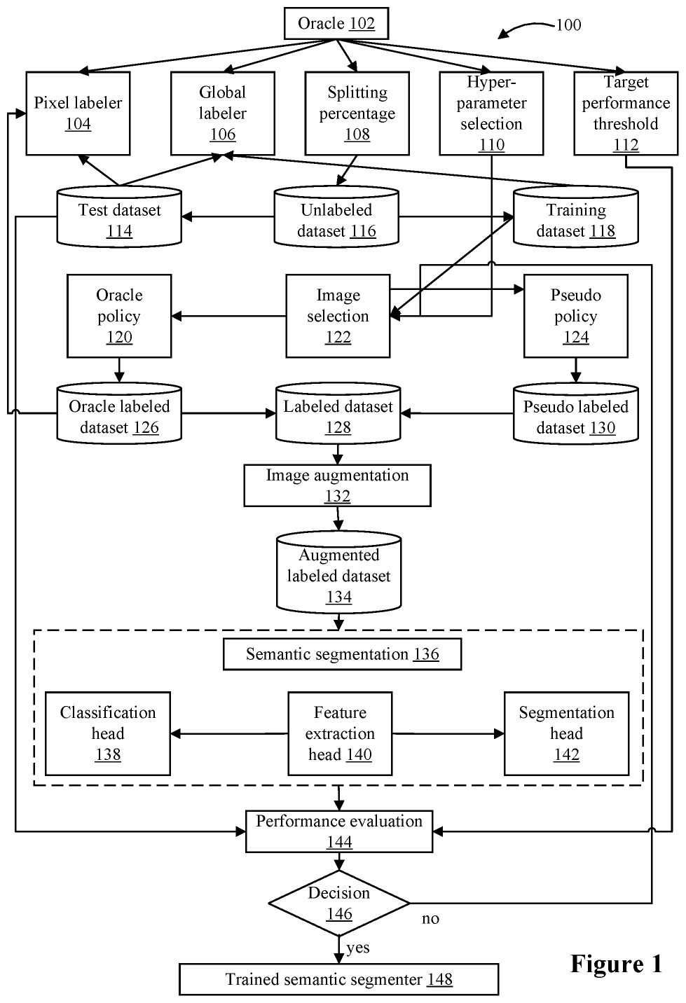

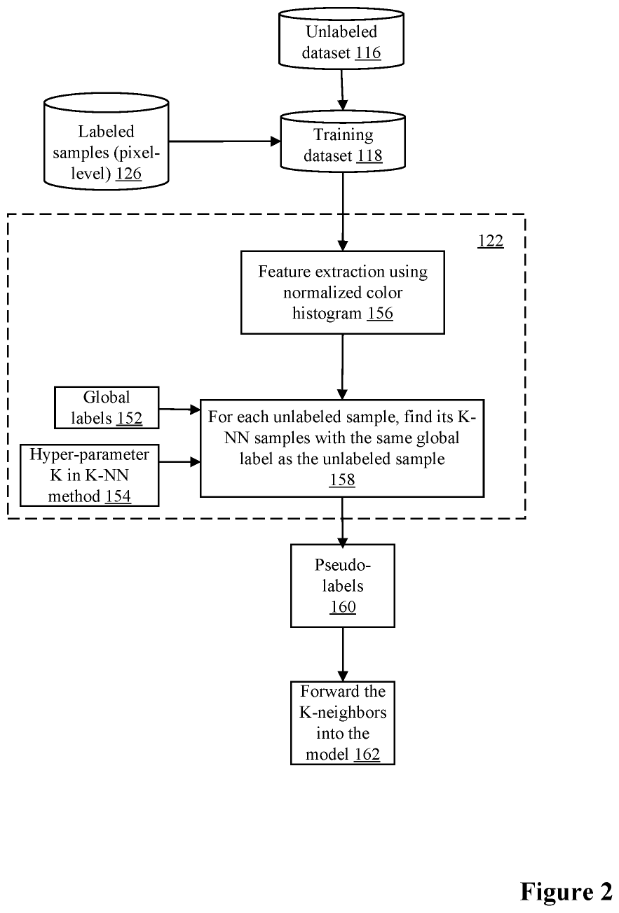

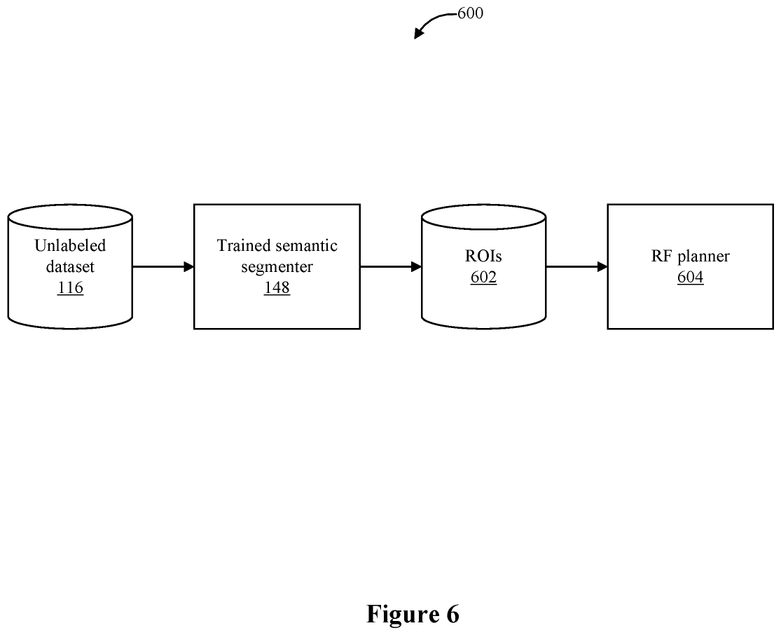

The method described herein has two parts, a training mode (illustrated in FIGS. 1 and 2) and an inference mode (illustrated in FIG. 6).

In the training mode, an oracle (which in most instances is a human expert but could be another system) provides a dataset of N-dimensional images (typically 2D or 3D images) of small cell hosting structures.

As shown in FIG. 1, the software asks the oracle to provide values for certain parameters and provide global annotation for all the images and pixel-level annotation for a test set.

After receiving the input from the oracle, the application starts training, as shown in FIG. 1, and asks the oracle for more pixel-level annotations during the active learning rounds. The application also performs image augmentation such as translation, rotation, skew, focus (blurring the images), blooming (over exposure of images), darkening under exposure of the images, shadowing (where shadows are cast due to time of day nearby structural shadows), blockage (where parts of the image are obfuscated by other objects such as structures, signs, vehicles, people), and coloring.

The application regularly compares the performance of the trained model with a target performance (confidence level) that should be achieved and that may be provided by the oracle according to a service level agreement (SLA). The training terminates when the target performance or confidence requirement is met and the trained model is saved to a disk.

In the inference mode (illustrated in FIG. 6), a dataset of fully unlabeled ND images, e.g. of small cell hosting structures, is provided as input to the model, i.e. there are no global annotations and no pixel-level annotations associated with the images.

An application loads the trained model that was saved during the training mode and runs it against the provided dataset. The model segments the regions of interests (e.g. poles, traffic signals, buildings, etc.) and labels each segmented region with the correct class.

An RF planner can then use the semantically segmented ND images of small cell hosting structures for a better installation planning.

The method described herein has several advantages over the previous work in the literature. Unlike standard deep active learning methods that primarily focus on the oracle acquisition function, the method described herein uses self-learning (label propagation method) as a second source of supervision to improve the segmentation with less pixel-wise annotated samples.

Deep weakly-supervised models are generally prone to high false positive rate, this issue is alleviated by using gradual pixel-wise supervision provided through an active learning framework with label propagation to train a deep convolutional architecture (segmentation head) that allows to output both classification and segmentation. Full resolution and more accurate masks are obtained compared to standard methods that are trained without pixel supervision and suffer from low resolution.

The level of self-learning can be adjusted to meet customer defined service level agreements, such as a 99% confidence level. This level adjustment is achieved through customer presented controls—including a number of “customer weakly-labeled images” used in the training phase and a desired service level agreement to be achieved.

It is worth noting that the method described herein can test and verify its own confidence level. This self-diagnostic capability enables this method to find use as a trusted customer tool.

Reference is now made to FIGS. 1 and 2.

In the method 100 illustrated in FIG. 1, a system interface asks the oracle 102 (e.g. a human expert that can provide accurate global and pixel-level labels for images) to provide a split percentage 108 for a training dataset 118 and test dataset 114, e.g. the oracle can set aside 10% of the unlabeled dataset 116 for testing and 90% for training. The oracle also provides global annotations (or labels) for all the images using the global labeler 106, which is an interface that facilitates image-level labeling through the system, and pixel-level annotations only for the test dataset 114 using the pixel labeler 104 which is an interface that facilitates pixel-level labeling through the system.

The oracle also enters a target performance threshold 112 which depends on the SLA, a.k.a. confidence level as well as hyper-parameter selection 110. The performance threshold 112 is compared against a dice index (the dice index is a statistic used to gauge the similarity of two samples, given two sets, X and Y, it is defined as DSC=(2|X∩Y|)/(|X|+|Y|) where |X| and |Y| are the cardinalities of the two sets (i.e. the number of elements in each set)) computed based on the results achieved by the model on the test set, which includes a value for the hyper-parameter 1, which determines the contribution of pseudo-labeled images during the training. Ep is a hyper-parameter entered by the oracle which determines the randomness of image selection for pseudo-labeling, i.e. the higher the value the more random the selection will be and the more exploration will have to be done as opposed to exploitation. ε₀ is an hyper-parameter entered by the oracle which determines the randomness of image selection for oracle-labeling. Both epsilon values guide the strategy for image selection. For example, if image selection can be made using Method #1 which picks images at random for labeling (i.e. it is nondeterministic) and Method #2 which only selects similar images for labeling (i.e. it is deterministic), then there are at least three options: always use Method #1, always use Method #2, sometimes (e.g. 20% of times) use Method #1 and sometimes (e.g. 80% of times) use Method #2. In the present disclosure and experiments diverse options are used. For example the last option can be used and the epsilon parameter specify the percentage of times that Method #1 and Method #2 are used. If epsilon is 0.2, then 20% of the times Method #1 is used and 80% of the times (i.e. 1-epsilon) Method #2 is used. Since there are two labeling schemes used (i.e. oracle labeling and automatic pseudo-labeling), there are two epsilons for each labeling scheme, namely co for oracle labeling and Ep for automatic pseudo-labeling. The experimentation presented further down, show a different manner in which these epsilon values can be set. The parameters A, co, Ep and the confidence level are numbers comprised between 0 and 1.

The test dataset 114 is sent to the performance evaluation 144 and the training dataset 118 is sent to the image selection 122. The image selection 122 decides which images will be selected for pseudo-labeling according to the pseudo-labeling policy and which images will be selected for oracle-labeling according to the oracle policy.

The performance evaluation 144 runs the trained model against the test set and calculates the dice index. The image selection consists of two parts, the oracle policy 120, and the pseudo policy 124.

The oracle policy 120 determines that pixel-wise unlabeled images with probability of εₒ will be selected randomly and with probability of 1-εₒ will be selected greedily (not at random) for oracle-labeling. The pseudo policy 124 determines that pixel-wise unlabeled images with probability of Ep will be selected randomly and with probability of 1-εₚ will be selected greedily (not at random) for pseudo-labeling.

It is important to note that both policies are sampling from the unlabeled (at the pixel-level) training dataset 118, the oracle policy 120 is sampling images for the oracle who labels them through the pixel labeler 104 while the pseudo policy 124 is sampling images for pseudo-labeling (no oracle involvement) which is an automatic label propagation mechanism. In both policies, a randomness is introduced to increase exploration, i.e. oracle and pseudo policies 120, 124 sample randomly with small fixed probabilities εₒ and εₚ respectively while they sample greedily with probabilities of 1-εₒ and 1-εₚ respectively.

A greedy policy is a policy that chooses a pixel-wise unlabeled sample whose distance from the labeled samples is minimum with respect to a distance metric. When the oracle policy chooses a pixel-wise unlabeled sample, the oracle labels the sample and adds it to the oracle labeled dataset 126. When the pseudo-labeling policy 124 chooses a pixel-wise unlabeled sample, the label of the nearest labeled sample is propagated to the pixel-wise unlabeled sample and the newly labeled sample is added to the pseudo-labeled dataset 130.

Turning to FIGS. 2-5, the pseudo-policy or pseudo-labeling 124 will be explained in more details. The pseudo-labeling is used to generate additional annotated samples beside the samples labeled by the oracle.

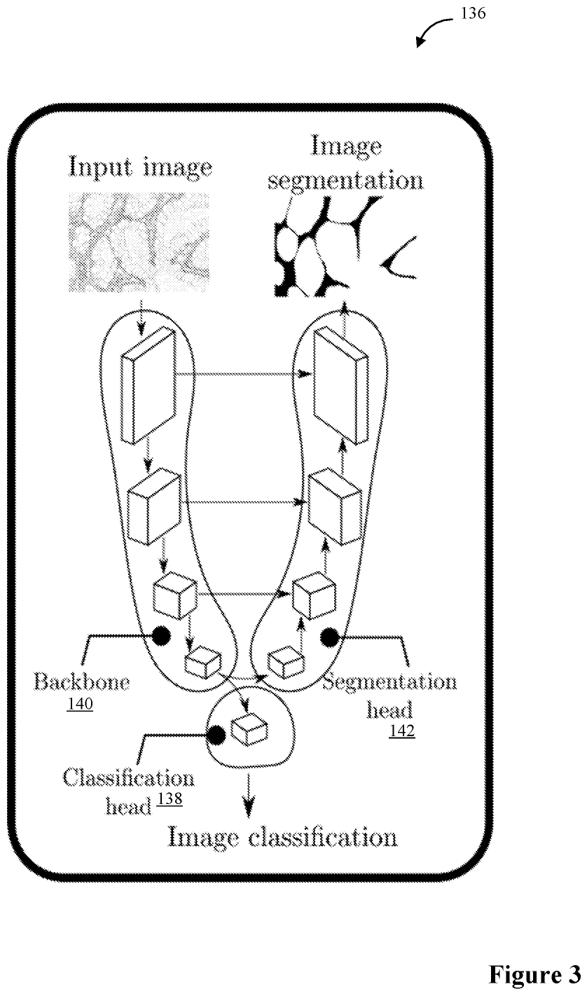

In FIG. 3, a hybrid architecture for classification and segmentation, with U-Net style, is shown. U-Net is a convolutional neural network that was developed for biomedical image segmentation at the Computer Science Department of the University of Freiburg. The architecture (semantic segmenter 136) is composed of a shared backbone 140 for feature extraction; a classification head 138 for the classification task; (3) and a segmentation head 142 for the segmentation task. The segmentation head 142 merges representations from the backbone while upscaling a feature map gradually to reach a full image resolution for a predicted mask.

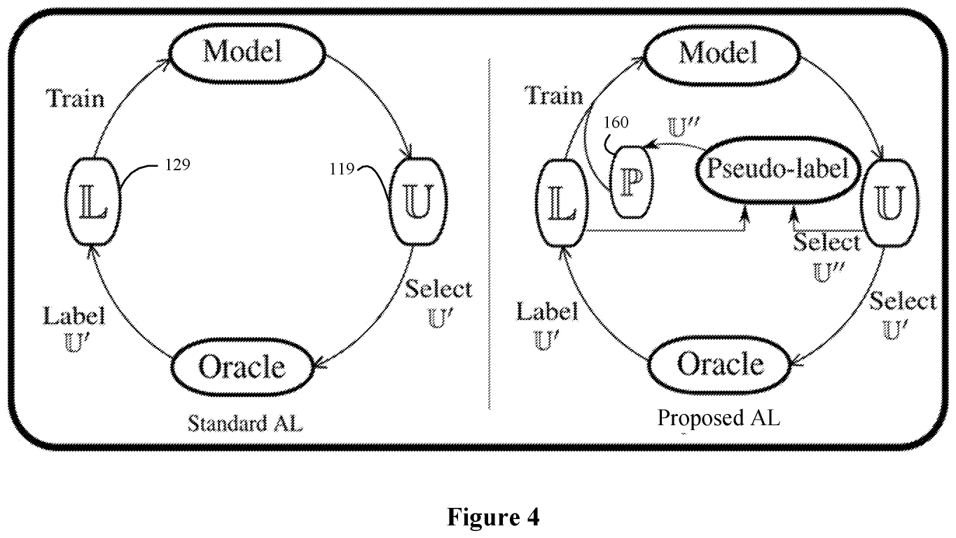

FIG. 4 illustrates a main difference between the method described herein and the standard active learning (AL) method. Usually, AL methods focus only on the acquisition function which is the core-driver of AL. However, the main drawback of the standard AL method is that it requires a lot of oracle-queries to achieve reliable and high performing models, which increases the annotation cost.

To reduce such cost, the method provided herein aims to reduce the oracle-queries while aiming for high performance through the use of a secondary source of annotation. Pseudo-annotation 124 of additional samples is obtained using self-learning.

While oracle-based annotation is generally accurate, the pseudo-annotation provided by the model can be considered as weak (noisy and less accurate). However, despite this inaccuracy, self-learning can provide a large boost to the performance as shown herein. In FIG. 4, U denotes unlabeled images 119, and L denotes labeled images 129. U′ are images selected to be labeled by an oracle while U″ are images selected to be labeled by the pseudo-annotation. P denotes pseudo-labelled images 160. It can be seen in this figure that the model is trained using images labeled by the oracle as well as images pseudo labeled by the model itself.

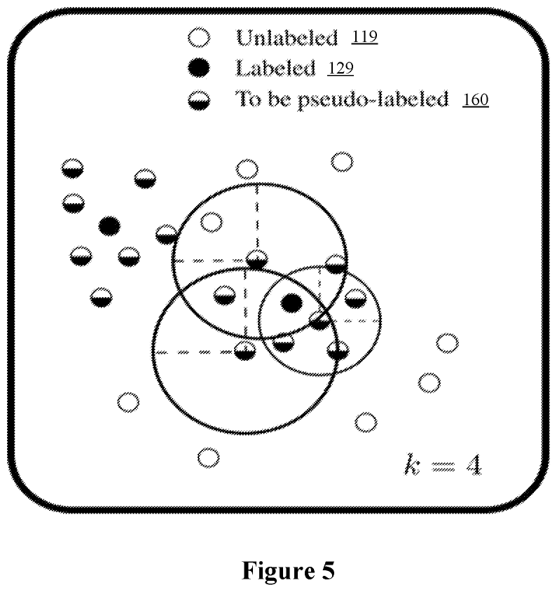

FIG. 5 illustrates how samples are obtained for pseudo-labelling. For each unlabeled sample 119, the algorithm checks if there is a nearby labeled sample 129. If yes, this sample is considered for pseudo-labeling 160.

FIG. 5 illustrates the k-nearest neighbors algorithm for selecting the subset U″ to be pseudo-labeled. In statistics, the k-nearest neighbors algorithm (K-NN) is a non-parametric classification method. In this work, K-NN is used to delineate a sample proximity based on a distance in a predefined feature space. The K-NN selection used herein is based on the assumption that unlabeled samples located nearby supervised samples are more likely to be assigned a good segmentation by the model. In order to measure a distance between two samples, a feature vector is first extracted from each image. Herein, a normalized color histogram (forming a probability distribution for each image) is considered as a generic and shape independent image feature vector. The Jensen-Shannon divergence between probability distributions is considered as a distance. To select U″, the algorithm goes through all unlabeled samples, and for each unlabeled sample, a distance is measured from other labeled samples from the same class.

It should be noted that the distance measure is made only between samples from the same class. For instance, it is better to pseudo-label image samples having ‘building’ in them that are located nearby labeled samples having ‘building’ in them as well. This is preferable simply because the model has already been trained with samples that contains ‘building’, giving it more chance to accurately segment unseen samples with ‘building’ in them. An unlabeled sample is considered for pseudo-labeling only if it has at least one labeled sample in the unlabeled sample K-NN perimeter, as illustrated in FIG. 5.

K-NN, or the set of k-nearest neighbors, is measured for each unlabeled sample. In the example of FIG. 5, using k=4 (k is a hyper-parameter indicating how many neighbors to consider, in this case after measuring the distance between all samples only the 4 closest are considered) allows |U″|=15. If K-NN is considered for each labeled sample (k=4) |U″|=8, where |.| is the number of elements in the set, i.e. set cardinal. It should be noted that K-NN is considered only between samples of the same class.

The assumption behind using K-NN for selection samples is that if a sample is labeled by the oracle, the model is more likely to provide good segmentation for nearby samples. It should be noted that one model can handle multiple classes at once. For example, in the experiments that have been run, a sample could have up to three classes: poles, buildings, vegetation. In additional experiments, up to 20 classes were used. Such models as described herein can handle a large number of classes.

To perform similarity comparisons, images need to have the same size (height and width). Since images may vary in size and using raw images to measure similarity is prone to large fluctuations by slight change in images, a normalized color histogram is used. For each image, the normalized color histogram is computed for each color channel. For RGB (red, green, blue) images, the histograms are averaged. Histograms are generic, shape independent, and fast to compute descriptors. Other descriptors could be used as well including more robust texture and color features. Since samples are represented by distributions, the Jensen-Shannon divergence is considered as a similarity measure. Other measures could be used such as L_p norm of difference between descriptors.

Referring again to FIG. 2, the image selection 122 and pseudo-labeling 124 are implemented using a self-learning method. Global labels 152 are available for all samples, labeled 126 or unlabeled 116. The method starts by performing, over the unlabeled samples 118, a selection 122 of a subset U″ to be pseudo-labeled by the trained model. As explained previously, instead of randomly selecting these samples, the method selects unlabeled samples that have at least one sample labeled by the oracle in their K-neighborhood. To do so, a similarity measure is computed between each unlabeled sample and the entire dataset. Unlabeled samples with at least one labeled sample in their K-NN are selected for pseudo-labeling. K-NN considers similarity only between samples having the same global label.

To select, 122, the images that will be pseudo-labelled 160, global labels 152 are assumed available for all samples. Feature extraction using normalized color histogram 156 is performed on all the training dataset. The feature extraction is used to perform similarity between the images. A hyper-parameter k 154 is used as input for the K-NN algorithm, which, for each unlabeled sample, finds the K nearest samples with the same global label as the unlabeled sample. In step 158, if among these K-NN samples there is at least one labeled sample, that unlabeled sample is considered for pseudo-labeling 160.

All samples in the dataset are labeled globally. A labeled sample is a sample that is labeled at the pixel-level. An unlabeled sample is a sample that is missing pixel-level label. A global label is a label that says what classes are in the image, for example ‘building’ is a class. A pixel-label of the class ‘building’ is a mask that indicates at pixel-level where the class ‘building’ is in the image.

Once a sample is selected to be pseudo-labeled, the sample is forwarded into the model to predict its segmentation. This produces a sample with pseudo-segmentation, i.e., segmentation obtained by the model and not the oracle. This sample will be added to the training set and used to increase its size, and it will be used for training. The pseudo-annotation is not completely accurate, but it helps improve the model segmentation performance.

These k nearest samples are the images that are pseudo-labeled 160 and are forwarded in the model, 162, to obtain pseudo-segmentation. Referring to FIG. 1, such samples with their pseudo-labels are added to the training labeled dataset 128 and used for training. This novel approach uses the model as a second source of supervision to increase the size of the training dataset.

One assumption here is that the model will acquire good knowledge over the labeled samples to allow it to provide good pseudo-labels for the samples nearby. This provides a second source of annotation for training the model. However, this second source of annotation is not accurate and prone to errors. To deal with this uncertainty in the annotation from this second source, the pseudo-labeled samples are considered differently in the training loss function, where their contribution in the model training is weighted using a lambda coefficient.

The custom training loss is a function that defines how the model is trained using the samples and their labels; and it is to be minimized. Training loss=E (labeled samples)+λ*E (pseudo_labeled samples) where E( ) is a function that measures the error of the model prediction compared to the provided annotation. The parameter A in the loss function controls the contribution of pseudo-labeled images. A small lambda is used when there is low confidence in the pseudo-labels while a high lambda is used when there is high confidence in the pseudo-labels. The value of lambda can be adjusted empirically using a validation set.

After pseudo-labelling is completed, the labeled dataset 128 is the union of the disjoint oracle labeled dataset 126 and pseudo-labeled dataset 130 where the algorithm keeps track of the selected pixel-wise unlabeled samples to enforce the disjointness condition. Next, the labeled dataset 128 is fed to the image augmentation 132 where randomized translational and rotational viewings of the images will be added for a robust training, the result is the augmented labeled dataset 134. The image augmentation 132 is therefore responsible for image augmentation operations such as translation, rotation, adding shadows, blurring, etc.

The augmented labeled dataset 134 is fed to the semantic segmentation 136 which consist of a feature extraction backbone 140, a classification head 138 and a segmentation head 142, as also illustrated in FIG. 3. The semantic segmentation 136 architecture allows to perform supervised classification learning using a classification head 138, and supervised segmentation learning using a dedicated head for segmentation 142. This is different from common architectures that are exclusively designed for one task and rarely for both.

The feature extraction head 140 is a residual convolutional neural network which consists of N-dimensional (ND) convolutions for ND images.

For example, for Google™ 3D street view images, the convolutions are 3-dimensional. The classification head 138 and segmentation head 142 are separate neural networks sharing the same feature extraction head backbone. Once trained and given an input image, this model 136 can classify the images, and provide full resolution of ROIs related to the predicted class.

The trained model 136 is sent to the performance evaluation 144 to be evaluated against the test dataset 114. Here, dice index is used as the evaluation metric. Multiple updates or versions of the model can be generated this way before the model meets the desired performance. Each time the model is evaluated and trained, its parameters (weights) are updated and the model should provide increasingly better performances. If the dice index of the best model is greater than the target performance threshold 112 then the training can be terminated (decision 146) and the trained semantic segmenter model 148 can be saved, otherwise the training continues and the oracle has to label more images until the desired target performance is reached.

This feedback loop is used for self-improvement.

In FIG. 6, the inference 600 is shown where the trained semantic segmenter 148 receives a set of (pixel-wise and global) unlabeled ND images (unlabeled dataset 116) and outputs the contours of ROIs 602 together with their predicted labels. Then, this information can be provided to an RF planning software 604 or to an expert.

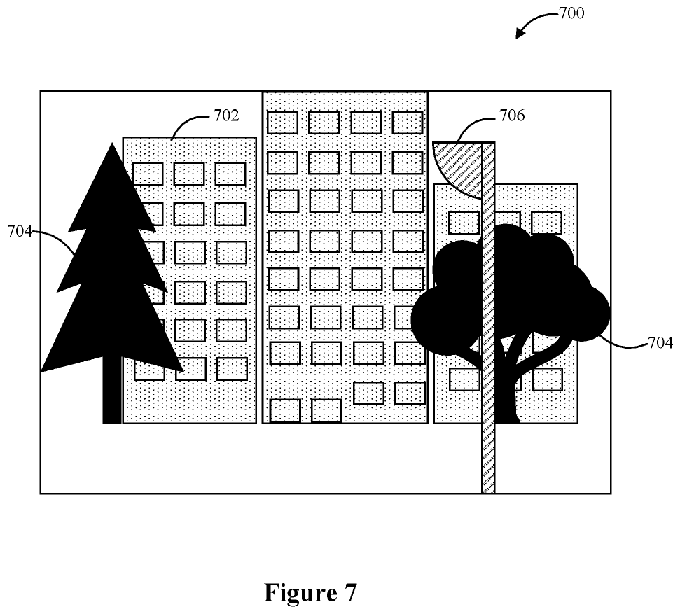

FIG. 7 is a schematic illustration of an example output image 700 with annotated ROIs 702, 704, 706 identified with a color or shading code and with other regions (e.g. background, not marked). In this example, black is used to identify vegetation 704, a dotted pattern is used to identify buildings 702, and a hatched pattern (diagonal stripes) is used to identify poles 706.

Usually, active learning (AL) methods focus only on the acquisition function which is the core-driver of AL. However, their main drawback is that they require a lot of oracle-queries to achieve reliable and high performing models which increases the annotation cost. To reduce such cost, the method presented herein aims at reducing the oracle-queries while aiming for high performance using a secondary source of annotation.

The method presented herein uses self-learning where the model provides pseudo-annotation of additional samples. While oracle-based annotation is accurate, the model's pseudo-annotation is usually weak (noisy and less accurate). However, despite this inaccuracy, self-learning can provide a large boost to performance.

The proposed general weakly supervised learning method described herein had been implemented to demonstrate its effectiveness in practice. The number of dimensions N was set to 2, i.e. 2D images and 2D convolutions were considered. For the oracle policy, the co was set to 1, i.e. the pixel-wise unlabeled samples for the oracle are selected randomly according to a uniform distribution. For the pseudo-labeling policy, the Ep was set to 0, i.e. pixel-wise unlabeled samples are not selected randomly but according to a greedy label propagation method. In particular, the K-NN method was chosen for label propagation and the Jensen-Shannon divergence was used between normalized color histograms of images as the distance metric.

A public dataset for street view scenes, named Cityscapes (https://www.cityscapes-dataset.com/), was considered, and only three classes were considered, i.e. poles, buildings and vegetation (trees and grass). For training, fourteen cities (2464 samples) were considered. For validation, four cities with total of 511 samples were considered. For the test set, the competition validation set of three cities with 500 samples was considered. Cities in each set were exclusive.

The experiments started by labeling five samples per class, and a sample per class afterwards. Thirty AL rounds were performed in total due to computation time. The experiments were repeated five times. The classification accuracy for the classification task and area under Dice index curve for the segmentation task are reported below. The method provided herein, was compared with five different methods:
1. Random selection (Random).
2. Entropy-based selection (Entropy).
3. Monte-Carlo dropout method (MC_Dropout).
4. Lower bound performance using weakly-supervised method (WSL).
5. Upper bound performance using fully pixel-wise supervised (Full_sup).

TABLE 1
Comparison of different classifications methods and average area under the curve and
standard deviation for Dice index performance over the Cityscapes test data set.

| | | Average area under |
|---|---|---|
| | | the Dice curve and |
| Method | Description | standard deviation |
| WSL | Not to be confused with the general WSL | 23.58 ± 7.36 |
| | methods. Here, WSL means no pixel-level | |
| | annotation (no oracle labeling and no active | |
| | learning), only global annotation plus CAM. | |
| Random | Both pixel-level and global annotations are | 41.61 ± 1.02 |
| | used, but oracle image selection (AL) is | |
| | completely random, i.e. ε₀ = 1, there is no | |
| | pseudo-labeling. | |
| Entropy | Both pixel-level and global annotations are | 43.20 ± 1.04 |
| | used, oracle image selection (AL) is | |
| | performed by choosing the images that have | |
| | the highest entropy (most uncertain), there is | |
| | no pseudo-labeling. | |
| MC_Dropout | Both pixel-level and global annotations are | 43.78 ± 1.26 |
| | used, but special functions (employing | |
| | Bayesian CNN for uncertainty estimation) are | |
| | used for oracle image selection (AL) that | |
| | selects the most uncertain samples, there is no | |
| | pseudo-labeling. | |
| Full_sup | Fully supervised, all images have both global | 69.43 ± 0.41 |
| | and pixel-level annotations. | |
| The method | Both pixel-level and global annotations are | 50.00 ± 0.22 |
| proposed | used, oracle image selection (AL) is | |
| herein | completely random, i.e. ε₀ = 1, but pseudo | |
| | image selection is completely greedy, i.e. | |
| | εₚ = 0, K-NN is used for pseudo-labeling. | |

In term of classification, an accuracy of 97.86+−0.11 was obtained over a test set. In term of segmentation, Table 1 shows the overall performance over thirty AL rounds.

The obtained results show that WSL does not provide accurate segmentation. In this case, the co-occurrence of all labels in the samples did not help such method. Note that thirty AL rounds allows to label (at the pixel-level) about 5% of the total training set.

Entropy and MC_Dropout methods yielded relatively similar performances. Using extra supervision, the method described herein ranked first using the same oracle-budget, demonstrating the advantage of self-learning (pseudo-labeling).

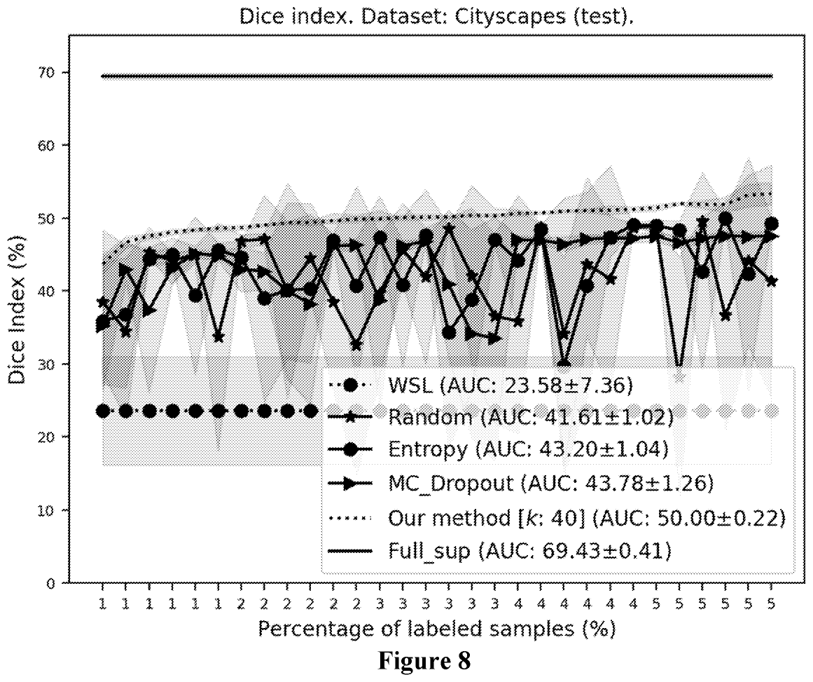

FIG. 8 illustrates the average Dice index of the proposed and baselines methods over the Cityscapes test dataset. FIG. 8 shows that the performance of the method described herein is better than the rest. Moreover, this method has less variance due to pseudo-labeling and the increased samples allow steady but slow increase of performance.

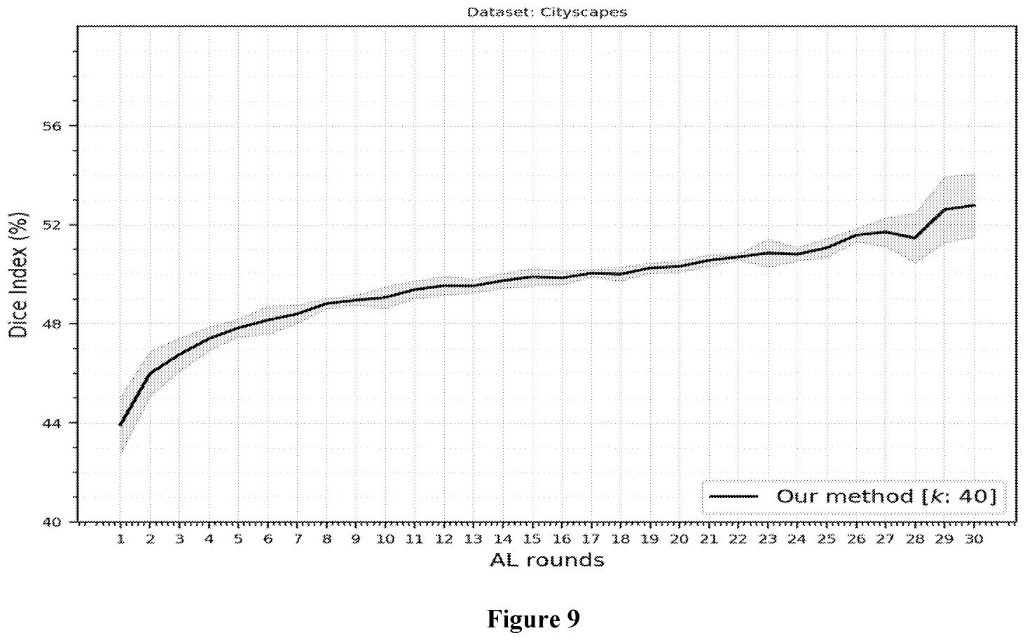

FIG. 9 illustrates the average Dice index over the pseudo-labeled samples of the method described herein in each AL round. FIG. 9 confirms the fact that the performance of the method described herein keeps increasing with adding more oracle-based samples.

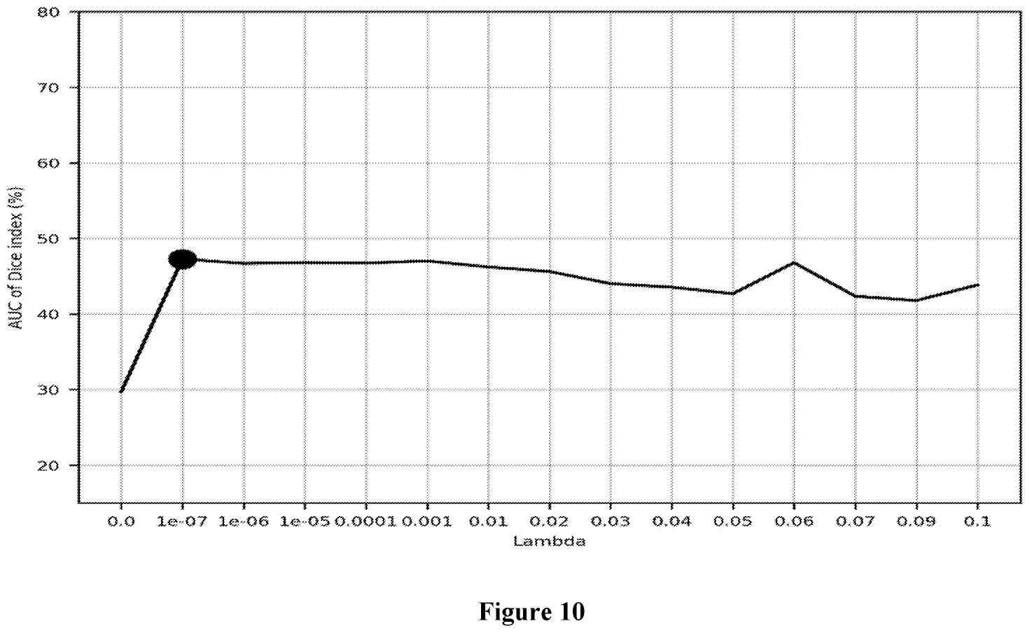

FIG. 10 illustrates an ablation study over the Cityscapes dataset (test dataset) over the hyper-parameter lambda (x-axis). Y-axis represents the area under the curve (AUC) of the Dice index (%) of five queries for one trial. The best performance is highlighted using a black dot, where lambda=1e-07 and AUC=47.26%. FIG. 10 shows the impact of λ on the performance, Δ=0 means no pseudo-labeled images are used. Following the experiments, using a small value such as λ=1e-07 is recommended.

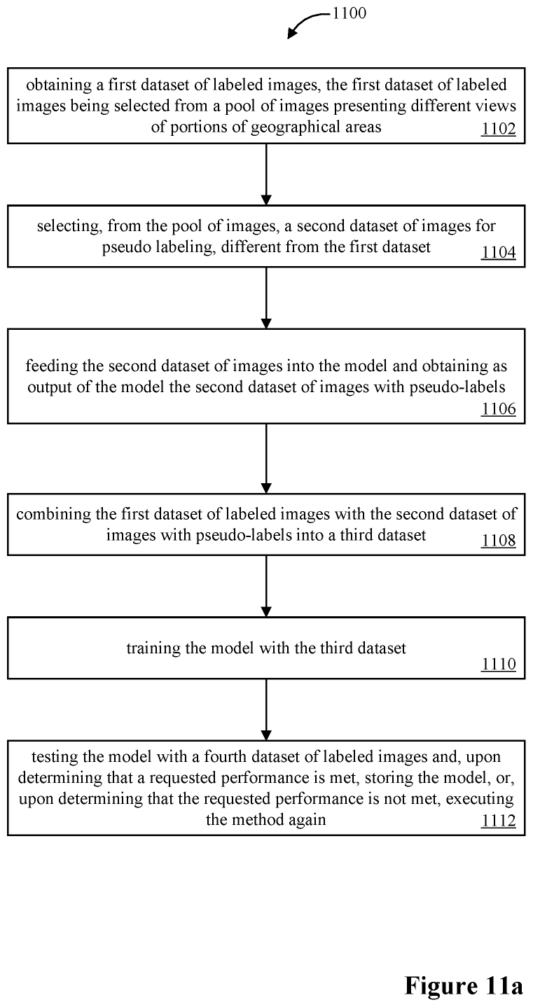

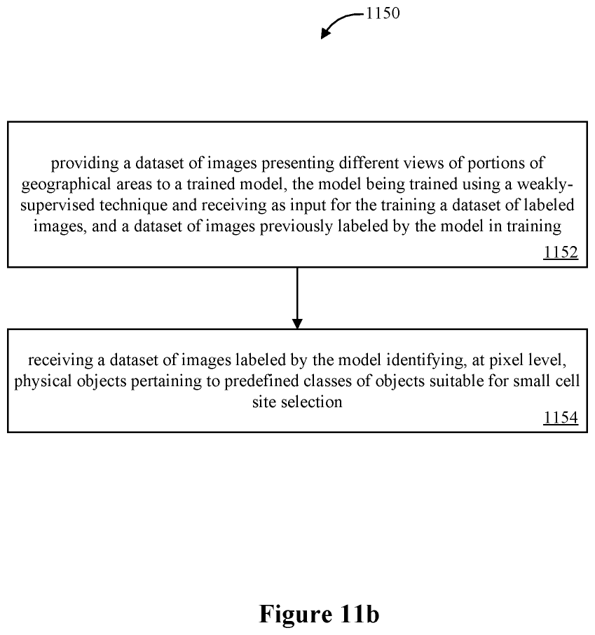

FIG. 11a illustrates a method 1100, or computer-implemented method, for training a model for labeling images for small cell site selection. The method comprises obtaining, step 1102, a first dataset of labeled images, the first dataset of labeled images being selected from a pool of images presenting different views of portions of geographical areas. The first dataset of labeled images may be labeled by an external source, such that the labeling is verified and trustable. The method comprises selecting, step 1104, from the pool of images, a second dataset of images for pseudo labeling, different from the first dataset. The method comprises feeding, step 1106, the second dataset of images into the model and obtaining as output of the model the second dataset of images with pseudo-labels. The method comprises combining, step 1108, the first dataset of labeled images with the second dataset of images with pseudo-labels into a third dataset. The method comprises training, step 1110, the model with the third dataset. The method comprises testing, step 1112, the model with a fourth dataset of labeled images and, upon determining that a requested performance is met, storing the model, or, upon determining that the requested performance is not met, executing the method again.

A first portion of the first and of the second datasets may be selected randomly and a second portion of the first and of the second datasets may be selected greedily. These portions may vary according to the dataset. Selecting an image greedily may comprise selecting a pixel-wise unlabeled image, among a plurality of pixel-wise unlabeled images, which has a minimum distance from a labeled image. The distance may be computed using a Jensen-Shannon divergence metric, normalized color histograms, and a k-nearest neighbors algorithm.

The model may be operative to identify, at pixel level, physical objects pertaining to predefined classes of objects suitable for small cell site selection. Identifying, at pixel level, may comprise generating class activation maps. The model may comprise a shared backbone for feature extraction from the images, a classification head for classifying objects pertaining to predefined classes of objects and a segmentation head for producing the class activation maps.

The method may further comprise, before the step of training, performing image augmentation operations on the images of the third dataset, the image augmentation operations being selected among: translation, rotation, skew, blurring, sharpening, lightening, darkening, shadowing, blockage, affine and projective transformations, noise addition and coloring.

Determining that the requested performance is met or not met may be done by comparing a performance threshold with a dice index (DSC) computed using DSC=(2|X ∩Y|)/(|X|+|Y|), where |X| and |Y| are cardinalities of both sets X and Y, X corresponding to the output of the model and Y corresponding to a ground truth. Ground truth refers to information that can be obtained by direct observation (i.e. empirical evidence).

FIG. 11b illustrates a method 1150 for obtaining labeled images for small cell site selection. The method comprises providing, step 1152, a dataset of images presenting different views of portions of geographical areas to a trained model, the model being trained using a weakly-supervised technique and receiving as input for the training a dataset of labeled images and a dataset of images previously labeled by the model in training. The method comprises receiving, step 1154, a dataset of images labeled by the model identifying, at pixel level, physical objects pertaining to predefined classes of objects suitable for small cell site selection.

Identifying, at pixel level, physical objects pertaining to predefined classes of objects suitable for small cell site selection may comprise identifying regions of interest (ROIs) segmented and labeled according to the classes of objects suitable for small cell site selection. The model may be trained according to the method illustrated in FIG. 11a.

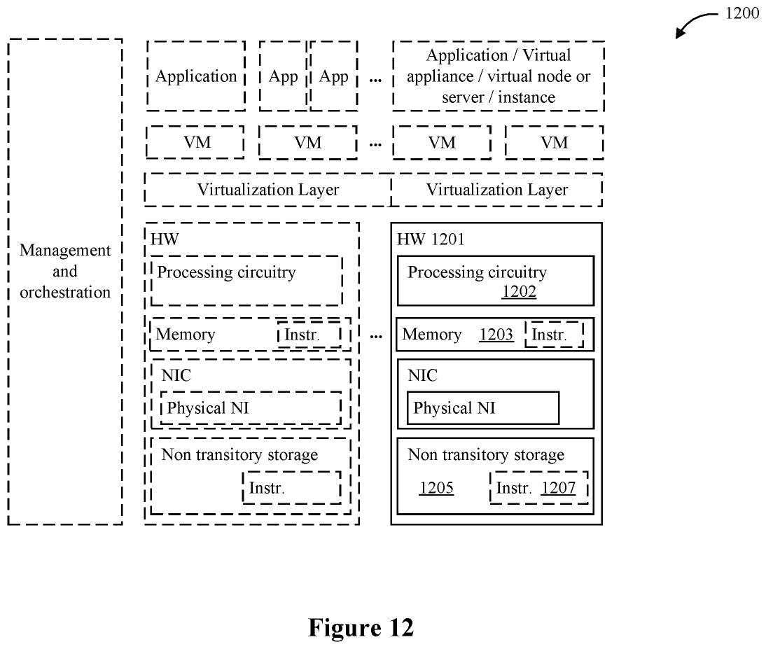

Referring to FIG. 12, there is provided a system 1200, which may implement a virtualization environment, in which functions and steps described herein can be implemented.

A virtualization environment (which may go beyond what is illustrated in FIG. 12), may comprise systems, networks, servers, nodes, devices, etc., that are in communication with each other either through wires or wirelessly. Some or all of the functions and steps described herein may be implemented as one or more virtual components (e.g., via one or more applications, components, functions, virtual machines or containers, etc.) executing on one or more physical apparatus in one or more networks, systems, environments, etc.

A virtualization environment provides hardware 1201 comprising processing circuitry 1202 and memory 1203. The memory can contain instructions executable by the processing circuitry whereby functions and steps described herein may be executed to provide any of the relevant features and benefits disclosed herein.

The hardware may also include non-transitory, persistent, machine readable storage media 1205 having stored therein software and/or instruction 1207 executable by processing circuitry to execute functions and steps described herein.

Referring to FIG. 12, there is provided a system 1200, or a hardware 1201, operative to train a model for labeling images for small cell site selection comprising processing circuits 1202 and a memory 1203, 1205. The memory contains instructions 1207 executable by the processing circuits 1202 whereby the system is operative to obtain a first dataset of labeled images, the first dataset of labeled images being selected from a pool of images presenting different views of portions of geographical areas. The system 1200, or a hardware 1201, is operative to select, from the pool of images, a second dataset of images for pseudo labeling, different from the first dataset. The system 1200, or a hardware 1201, is operative to feed the second dataset of images into the model and obtain as output of the model the second dataset of images with pseudo-labels. The system 1200, or a hardware 1201, is operative to combine the first dataset of labeled images with the second dataset of images with pseudo-labels into a third dataset. The system 1200, or a hardware 1201, is operative to train the model with the third dataset. The system 1200, or a hardware 1201, is operative to test the model with a fourth dataset of labeled images and, upon determining that a requested performance is met, storing the model, or, upon determining that the requested performance is not met, executing the method again.

A first portion of the first and second datasets may be selected randomly and a second portion of the first and second datasets may be selected greedily. These portions may vary according to the dataset. Selecting an image greedily may comprise selecting a pixel-wise unlabeled image, among a plurality of pixel-wise unlabeled images, which has a minimum distance from a labeled image. The distance may be computed using a Jensen-Shannon divergence metric, normalized color histograms, and a k-nearest neighbors algorithm.

The model may be operative to identify, at pixel level, physical objects pertaining to predefined classes of objects suitable for small cell site selection. Identifying, at pixel level, may comprises generating class activation maps. The model may comprise a shared backbone for feature extraction from the images, a classification head for classifying objects pertaining to predefined classes of objects and a segmentation head for producing the class activation maps.

The system 1200, or hardware 1201, may be further operative to perform image augmentation operations on the images of the third dataset, the image augmentation operations being selected among: translation, rotation, skew, blurring, sharpening, lightening, darkening, shadowing, blockage, affine and projective transformations, noise addition and coloring.

Determining that the requested performance is met or not met may be done by comparing a performance threshold with a dice index (DSC) computed using DSC=(2|X ∩Y|)/(|X|+|Y|), where |X| and |Y| are cardinalities of both sets X and Y, X corresponding to the output of the model and Y corresponding to a ground truth.

There is provided a system 1200, or a hardware 1201, operative to obtain labeled images for small cell site selection. The system 1200, or hardware 1201, comprises processing circuits 1202 and a memory 1203, 1205. The memory contains instructions 1207 executable by the processing circuits 1202 whereby the system 1200 or hardware 1201 is operative to provide a dataset of images presenting different views of portions of geographical areas to a trained model, the model being trained using a weakly-supervised technique and receiving as input for the training a dataset of labeled images and a dataset of images previously labeled by the model in training. The system 1200 or hardware 1201 is operative to receive a dataset of images labeled by the model identifying, at pixel level, physical objects pertaining to predefined classes of objects suitable for small cell site selection.

Identifying, at pixel level, physical objects pertaining to predefined classes of objects suitable for small cell site selection may comprise identifying regions of interest (ROIs) segmented and labeled according to the classes of objects suitable for small cell site selection.

There is provided a non-transitory computer readable media 1205 having stored thereon instructions 1207 for training a model for labeling images for small cell site selection, the instructions comprising any of the steps described herein.

There is provided a non-transitory computer readable media 1205 having stored thereon instructions 1207 for obtaining labeled images for small cell site selection, the instructions comprising any of the steps described herein.

Modifications will come to mind to one skilled in the art having the benefit of the teachings presented in the foregoing description and the associated drawings. Therefore, it is to be understood that modifications, such as specific forms other than those described above, are intended to be included within the scope of this disclosure. For example, the methods described herein may be applicable to, and used for, applications others than small cell site selection. The previous description is merely illustrative and should not be considered restrictive in any way. The scope sought is given by the appended claims, rather than the preceding description, and all variations and equivalents that fall within the range of the claims are intended to be embraced therein. Although specific terms may be employed herein, they are used in a generic and descriptive sense only and not for purposes of limitation.

1. A method for training a model for labeling images comprising:
obtaining a first dataset of labeled images, the first dataset of labeled images being selected from a pool of images presenting different views of portions of geographical areas;
selecting, from the pool of images, a second dataset of images for pseudo labeling, different from the first dataset;
feeding the second dataset of images into the model and obtaining as output of the model the second dataset of images with pseudo-labels;
combining the first dataset of labeled images with the second dataset of images with pseudo-labels into a third dataset;
training the model with the third dataset; and
testing the model with a fourth dataset of labeled images and, upon determining that a requested performance is met, storing the model, or, upon determining that the requested performance is not met, executing the method again.

2. The method of claim 1, wherein a first portion of the first dataset is selected randomly, and a second portion of the first dataset is selected greedily, wherein selecting an image greedily comprises selecting a pixel-wise unlabeled image, among a plurality of pixel-wise unlabeled images, which has a minimum distance from a labeled image, and wherein the distance is computed using a Jensen-Shannon divergence metric, normalized color histograms, and a k-nearest neighbors algorithm.

3. The method of claim 1, wherein a first portion of the second dataset is selected randomly, and a second portion of the second dataset is selected greedily, wherein selecting an image greedily comprises selecting a pixel-wise unlabeled image, among a plurality of pixel-wise unlabeled images, which has a minimum distance from a labeled image, and wherein the distance is computed using a Jensen-Shannon divergence metric, normalized color histograms, and a k-nearest neighbors algorithm.

4. (canceled)

5. (canceled)

6. The method of claim 1, wherein the model is operative to identify, at pixel level, physical objects pertaining to predefined classes of objects.

7. The method of claim 6, wherein identifying, at pixel level, comprises generating class activation maps.

8. The method of claim 7, wherein the model comprises a shared backbone for feature extraction from the images, a classification head for classifying objects pertaining to predefined classes of objects and a segmentation head for producing the class activation maps.

9. The method of claim 1, further comprising, before the step of training, performing image augmentation operations on the images of the third dataset, the image augmentation operations being selected among: translation, rotation, skew, blurring, sharpening, lightening, darkening, shadowing, blockage, affine and projective transformations, noise addition and coloring.

10. The method of claim 1, wherein determining that the requested performance is met or not met is done by comparing a performance threshold with a dice index (DSC) computed using DSC=(2|X ∩Y|)/(|X|+|Y|), where |X| and |Y| are cardinalities of both sets X and Y, X corresponding to the output of the model and Y corresponding to a ground truth.

11. A method for obtaining labeled images, comprising:
providing a dataset of images presenting different views of portions of geographical areas to a trained model, the model being trained using a weakly-supervised technique and receiving as input for the training a dataset of labeled images and a dataset of images previously labeled by the model in training; and
receiving a dataset of images labeled by the model identifying, at pixel level, physical objects pertaining to predefined classes of objects.

12. The method of claim 11, wherein identifying, at pixel level, physical objects pertaining to predefined classes of objects comprises identifying regions of interest (ROIs) segmented and labeled according to the classes of objects.

13. The method of claim 11, wherein the model is trained by:
obtaining a first dataset of labeled images, the first dataset of labeled images being selected from a pool of images presenting different views of portions of geographical areas;
selecting, from the pool of images, a second dataset of images for pseudo labeling, different from the first dataset;
feeding the second dataset of images into the model and obtaining as output of the model the second dataset of images with pseudo-labels;
combining the first dataset of labeled images with the second dataset of images with pseudo-labels into a third dataset;
training the model with the third dataset; and
testing the model with a fourth dataset of labeled images and, upon determining that a requested performance is met, storing the model, or, upon determining that the requested performance is not met, executing the method again.

14. A system operative to train a model for labeling images comprising processing circuits and a memory, the memory containing instructions executable by the processing circuits whereby the system is operative to:
obtain a first dataset of labeled images, the first dataset of labeled images being selected from a pool of images presenting different views of portions of geographical areas;
select, from the pool of images, a second dataset of images for pseudo labeling, different from the first dataset;
feed the second dataset of images into the model and obtain as output of the model the second dataset of images with pseudo-labels;
combine the first dataset of labeled images with the second dataset of images with pseudo-labels into a third dataset;
train the model with the third dataset; and
test the model with a fourth dataset of labeled images and, upon determining that a requested performance is met, storing the model, or, upon determining that the requested performance is not met, executing the method again.

15. The system of claim 14, wherein a first portion of the first dataset is selected randomly, and a second portion of the first dataset is selected greedily, wherein selecting an image greedily comprises selecting a pixel-wise unlabeled image, among a plurality of pixel-wise unlabeled images, which has a minimum distance from a labeled image, and wherein the distance is computed using a Jensen-Shannon divergence metric, normalized color histograms, and a k-nearest neighbors algorithm.

16. The system of claim 14, wherein a first portion of the second dataset is selected randomly, and a second portion of the second dataset is selected greedily, wherein selecting an image greedily comprises selecting a pixel-wise unlabeled image, among a plurality of pixel-wise unlabeled images, which has a minimum distance from a labeled image, and wherein the distance is computed using a Jensen-Shannon divergence metric, normalized color histograms, and a k-nearest neighbors algorithm.

17. (canceled)

18. (canceled)

19. The system of claim 14, wherein the model is operative to identify, at pixel level, physical objects pertaining to predefined classes of objects.

20. The system of claim 19, wherein identifying, at pixel level, comprises generating class activation maps.

21. The system of claim 20, wherein the model comprises a shared backbone for feature extraction from the images, a classification head for classifying objects pertaining to predefined classes of objects and a segmentation head for producing the class activation maps.

22. The system of claim 14, further operative to perform image augmentation operations on the images of the third dataset, the image augmentation operations being selected among: translation, rotation, skew, blurring, sharpening, lightening, darkening, shadowing, blockage, affine and projective transformations, noise addition and coloring.

23. The system of claim 14, wherein determining that the requested performance is met or not met is done by comparing a performance threshold with a dice index (DSC) computed using DSC=(2|X ∩Y|)/(|X|+|Y|), where |X| and |Y| are cardinalities of both sets X and Y, X corresponding to the output of the model and Y corresponding to a ground truth.

24. The system of claim 14, further operative to:
provide a dataset of images presenting different views of portions of geographical areas to a trained model, the model being trained using a weakly-supervised technique and receiving as input for the training a dataset of labeled images and a dataset of images previously labeled by the model in training; and
receive a dataset of images labeled by the model identifying, at pixel level, physical objects pertaining to predefined classes of objects.

25. (canceled)

26. (canceled)

27. (canceled)
